(19) United States
(12) Patent Application Publication
(10) Pub. No.: US 2025/0295520 A1
(43) Pub. Date: Sep. 25, 2025

(54) ELASTOMERIC ARTICLES WITH IMPROVED PROPERTIES

(71) Applicant: Church & Dwight Co., Inc., Princeton, NJ (US)

(72) Inventors: Andrew GUTTENTAG, Robbinsville, NJ (US); Sujata Sundara RAJAN, Belle Mead, NJ (US); Andrew LITZENBERGER, Pennington, NJ (US); Timothy SNOWDEN, Howell, NJ (US)

(73) Assignee: Church & Dwight Co., Inc., Princeton, NJ (US)

(21) Appl. No.: 18/864,395

(22) PCT Filed: May 10, 2023
(86) PCT No.: PCT/IB2023/054834

§ 371 Date: Nov. 8, 2024

Related U.S. Application Data
(60) Provisional application No. 63/340,549, filed on May 11, 2022.

(51) Int. Cl. A61F 6/04; C08K 3/011; C08K 3/38; C08L 7/02

(52) U.S. Cl. CPC A61F 6/04; C08K 3/011; C08K 3/38; C08L 7/02; C08K 2003/385; C08K 2201/001

(57) ABSTRACT

The present disclosure provides compositions and products formed therefrom. In particular, the disclosure provides elastomeric articles, such as gloves and condoms, that can be prepared utilizing a polymer latex composition that includes particles of boron nitride. The elastomeric articles can exhibit desired properties, such as tensile strength, lubricity, and thermal conductivity. The disclosure further provides methods of preparing elastomeric articles using polymer latex compositions including boron nitride particles.

### FIELD OF THE DISCLOSURE

The present disclosure relates to polymer compositions and products that are formed from the polymer compositions, such as elastomeric articles (e.g., thin-walled products, such as gloves and condoms). The elastomeric articles particularly can exhibit excellent mechanical properties, such as tensile strength and lubricity, as well as excellent thermal conductivity, which is effective to impart the articles with improved sensory properties, such as perception of coolness, warmth, and smoothness.

### BACKGROUND

Natural rubber, which is comprised primarily of cis-1,4-polyisoprene, is well known for use in making thin-film, elastomeric articles, such as surgical gloves, balloons, condoms, and the like. Various, synthetic polymers have likewise been known for use in preparation of such articles. Elastomeric, thin-film articles that are configured for bodily contact can exhibit a “cold” or “dry” feeling to the user, and the lack of a more natural feel to the elastomeric articles can dissuade from the use thereof. Accordingly, there remains a need in the field for elastomeric articles that can provide the desired tensile properties of similar elastomeric articles while also exhibiting additional properties that make the articles more desirable for personal use.

### SUMMARY OF THE DISCLOSURE

The present disclosure provides compositions of polymeric materials and products made therefrom. The products may include any material that is useful when provided in the form of a thin film that is elastomeric and exhibits suitable mechanical and thermal properties, such as gloves, condoms, and similar articles.

In one or more embodiments, the present disclosure can provide elastomeric articles comprising at least one layer of a polymer latex composition. In particular, the polymer latex composition from which the article is formed can include a polymeric component and boron nitride particles. The article also can be defined in relation to specific properties that arise from the nature of the composition used in preparing the article. For example, the elastomeric article at a thickness of about 0.1 mm or less can exhibit a tensile strength of about 20 MPa or greater when measured in accordance with ASTM D412. For example, the elastomeric article at a thickness of about 0.1 mm or less can exhibit a thermal conductivity that is at least 5% greater than the thermal conductivity of an elastomeric article that does not include the boron nitride particles but is otherwise substantially identical in composition. In further embodiments, the article can be further defined in relation to any one or more of the following statements, which statements are expressly understood as being combinable in any number and order.

The elastomeric article can be a condom.

The polymeric component can comprise natural rubber.

The polymeric component can comprise a synthetic polymer.

The polymeric component can comprise a polyisoprene or polystyrene-polyisoprene-polystyrene (SIS) copolymer.

The boron nitride particles can be configured as platelets.

The boron nitride particles can be configured as spheroids.

The boron nitride particles can have an average size of about 1 micron to about 25 microns.

The polymer latex composition can comprise about 0.1 to about 20 parts per hundred (phr) of the boron nitride particles based on the weight of the polymeric component.

The boron nitride particles can be substantially uniformly distributed in the at least one layer of the polymer latex composition.

The tensile strength of the elastomeric article can be greater than a tensile strength of an elastomeric article that does not include the boron nitride particles but is otherwise substantially identical in composition.

The elastomeric article can exhibit a lubricity that is greater than the lubricity of an elastomeric article that does not include the boron nitride particles but is otherwise substantially identical in composition.

The thermal conductivity can be defined as being a thru-plane thermal conductivity measured according to ASTM 1530.

The thermal conductivity can be defined as being an in-plane thermal conductivity measured according to ISO 22007-2.

The polymer latex composition can be compounded with one or both of a crosslinking agent and a cure accelerator.

The polymer latex composition further can comprise one or more of a surfactant, a catalyst, an antioxidant, a viscosity modifier, a filler, and a smoothing agent.

In one or more embodiments, the present disclosure can relate a method for improving thermal conductivity of a condom. In particular, the method can comprise forming the condom from a polymer latex composition that includes boron nitride particles so as to provide the condom with at least one layer of the polymer latex composition that has the boron nitride particles distributed within. The method also can be defined in relation to specific properties that arise from the nature of the composition used in the method of preparing the article. For example, the condom can exhibit a thermal conductivity that is at least 5% greater than the thermal conductivity of a condom that does not include the boron nitride particles but is otherwise substantially identical in composition. In further embodiments, the method can be further defined in relation to any one or more of the following statements, which statements are expressly understood as being combinable in any number and order.

The boron nitride particles can be included in the polymer latex composition in a substantially platelet-shaped form.

The boron nitride particles can be included in the polymer latex composition in a substantially spheroid shape.

The boron nitride particles can be substantially in the form of a plurality of agglomerates of smaller particles when included in the polymer latex composition.

### BRIEF DESCRIPTION OF THE DRAWING

FIG. 1A provides images taken with an optical of a natural rubber latex film (left) and a natural rubber latex film including boron nitride particles at 2.5 phr concentration (right).microscope in transmission mode at 7× magnification

FIG. 1B provides images taken with an optical microscope in reflectance mode at 90× magnification of a natural rubber latex film (left) and a natural rubber latex film including boron nitride particles at 2.5 phr concentration.

FIG. 2 provides scanning electron microscope (SEM) images at 300× (top row) and 3000× (bottom row) of a natural rubber latex film (left column) and a natural rubber latex film including boron nitride particles at 2.5 phr concentration (right column).

FIG. 3 provides a graph showing strain measurements of an elastomeric article made from natural rubber latex (NRL) as a control and elastomeric articles made from NRL and including varying dosings of boron nitride (BN) particles as test articles, the graph illustrating variances in strain as a factor of the BN content included.

FIG. 4 provides a graph showing tensile strength measurements taken using the dumbbell testing method for an elastomeric article made from NRL as a control and elastomeric articles made from NRL and including varying dosings of BN particles as test articles, the graph illustrating variances in tensile strength as a factor of the BN content included.

FIG. 5 provides a graph showing measured force required to move a slide across an elastomeric article made from NRL as a control and an elastomeric article made from NRL and including 2.5 parts per hundred rubber (phr) of BN particles.

FIG. 6 provides a graph showing calculated lubricity values for an elastomeric article made from NRL as a control and an elastomeric article made from NRL and including 2.5 phr of BN particles.

FIG. 7 provides a graph showing calculated lubricity values for an elastomeric article made from NRL as a control and an elastomeric article made from NRL and including varying dosings of BN particles as test articles.

FIG. 8 provides a graph showing lubricity values averaged from 300 individual readings for an elastomeric article made from NRL as a control and an elastomeric article made from NRL and including varying dosings of BN particles as test articles.

FIG. 9 provides a graph showing thru-plane thermal conductivity of an elastomeric article formed from NRL as a control and an elastomeric article formed from NRL including 10% by weight BN as a test article.

FIG. 10 provides a graph showing in-plane thermal conductivity of an elastomeric article formed from NRL as a control and an elastomeric article formed from NRL including 10% by weight BN as a test article.

FIG. 11 provides a graph showing average thermal conductivity of an elastomeric article including BN according to an example embodiment of the present disclosure against average thermal conductivity of four comparative elastomeric articles.

### DETAILED DESCRIPTION OF THE DISCLOSURE

The invention now will be described more fully hereinafter through reference to various embodiments. These embodiments are provided so that this disclosure will be thorough and complete, and will fully convey the scope of the invention to those skilled in the art. Indeed, the invention may be embodied in many different forms and should not be construed as limited to the embodiments set forth herein: rather, these embodiments are provided so that this disclosure will satisfy applicable legal requirements. As used in the specification, and in the appended claims, the singular forms “a”, “an”, “the”. include plural referents unless the context clearly dictates otherwise.

The present disclosure relates articles comprising polymer latex compositions. The articles particularly can be elastomeric articles and. even further, the articles may be provided as thin films. As used herein, a “thin” film can be a layer of the polymer latex composition (e.g., a sheet) that has a thickness of 0.5 mm or less and. more particularly. 0.3 mm or less. Elastomeric articles according to the present disclosure configured for specific uses as described herein specifically can have a thickness of about 0.3 mm or less. about 0.2 mm or less. or about 0.1 mm or less, such as about 0.01 mm to about 0.3 mm, about 0.02 mm to about 0.2 mm, about 0.03 mm to about 0.15 mm, about 0.04 mm to about 0.11 mm. or about 0.05 mm to about 0.1 mm.

An elastomeric article according to the present disclosure, while comprising a thin film, can be provided in a variety of shapes and configurations. In particular, the present disclosure encompasses elastomeric articles that are configured as condoms, gloves, finger cots, and similar items of manufacture. In such articles, substantially the entirety of the article is formed from the polymer latex composition. It is understood, however, that the present disclosure also encompasses other articles that, while including one or more thin film layers formed from the polymer latex composition, are not necessarily formed completely from the polymer latex composition.

The polymer latex compositions utilized in the elastomeric articles can utilize natural rubber as the polymeric component thereof. In some embodiments, a synthetic rubber can be used as the polymeric component. Non-limiting examples of synthetic rubber polymers that may be used include synthetic polyisoprene, synthetic poly(styrene-isoprene-styrene) (“SIS”), intermediate modulus (“IM”) styrene ethylene butylene styrene (“SEBS”), high modulus (“HM”) SEBS, water-based polyurethane, nitrile rubber (e.g., acrylonitrile butadiene rubber, or “NBR”), styrene-co-butadiene, styrene-co-isoprene, triblock copolymers, such as styrene-block-butadiene and block styrene (SBS), and similar, synthetic polymers in the form of homopolymers and/or co-polymers. In addition to the above, the polymer latex compositions may comprise a combination of a natural rubber and one or more synthetic rubbers. Likewise, natural rubber may be expressly excluded in favor of one or more synthetic rubber, or synthetic rubbers may be expressly excluded in favor of natural rubber.

In one or more embodiments, the present disclosure can provide elastomeric articles comprising at least one layer of a polymer latex composition. A single layer can be the result of a single dipping of a mold into a liquid form of the polymer latex composition. In other embodiments, a single layer can be the result of a plurality of dippings of the mold so that the thickness of the layer increases with each successive dipping. In such embodiments, it is understood that the film resulting from each successive dipping will adhere or otherwise bond to the underlying film so as to result in a single layer (i.e., there will be no delamination of films).

In addition to the polymeric component, the polymer latex composition of the present disclosure can include an amount of at least one particulate component effective to improve properties of the formed. elastomeric article, such as mechanical properties, thermal properties, and sensory properties. Boron nitride can be particularly useful in this regard. As further described herein. elastomeric articles prepared with a polymer latex composition including boron nitride particles exhibit improved texture and improved thermal conductivity without loss in mechanical properties (such as tensile strength) or even improved mechanical properties. The improved texture can provide superior comfort for a user, particularly when the elastomeric article is configured for bodily contact, such as with condoms. Rubber latex articles are poor conductors of heat, and the addition of the boron nitride particles provides improved heat transfer by the elastomeric articles of the present disclosure.

Boron nitride particles can be utilized in a variety of shapes and sizes. In some embodiments, the boron nitride particles particularly are configured as platelets. A “platelet” is intended to encompass a three-dimensional shape that is flattened so that particle thickness is less than the remaining dimensions (e.g., length and width). Hexagonal boron nitride particles can be particularly useful. The choice of boron nitride particle shape can be particularly beneficial for tuning thermal conductivity of the formed, elastomeric article. In some embodiments, the boron nitride particles can be configured as spheroids and thus may be substantially spherical without the necessity for being perfectly spherical. In some embodiments, the boron nitride particles can be configured as flakes. The particles likewise may be irregularly shaped. Any particle shape may be utilized in various embodiments of the disclosure, although specific shapes may be preferred in some embodiments, and specific shapes may likewise be expressly excluded in some embodiments. It is also understood that the particles may be substantially individualized. In particular embodiments, however, the particles may be in the form of agglomerates of smaller particles. A plurality of the agglomerates may thus be used solely or in combination with individualized particles.

The size of the boron nitride particles can vary, and the size of particles utilized can relate to the desired thickness of the elastomeric article to be made. In some embodiments, the particle size may be limited only by the ability to include the particles without otherwise making the end product ineffective for the desired use. Particle size, for example can be up to about 100 microns. up to about 75 microns, up to about 50 microns, or up to about 25 microns. Particle size, for example, may be at least 1 micron, at least 2 microns, at least 5 microns, at least 10 microns, or at least 20 microns. All ranges bound by any selection of the lower and upper sized noted above are expressly included. In some embodiments. it may be desirable to utilize a specifically smaller size range. For example, a maximum dimension can be in the range of about 1 micron to about 25 microns, about 2 microns to about 20 microns, about 3 microns to about 15 microns, or about 4 microns to about 12 microns. When the boron nitride particles are configured as platelets, the noted ranges can relate to the length and width of the platelets with the understanding that the thickness of the platelets will be less than the smallest dimension of length and width. When the boron nitride particles are configured as spheroids or are irregularly shaped, the noted ranges can define a mean particle size or a median particle size.

The content of boron nitride particles in the polymer latex composition can vary. In some embodiments, the particle concentration may be adjustable to achieve specifically desired properties in the article to be prepared from the polymer latex composition. Particle concentration, for example can be up to about 20 parts her hundred rubber (phr), up to about 15 phr, up to about 10 phr, up to about 8 phr, or up to about 6 phr. Particle concentration. for example, may be at least 0.01 phr, at least 0.05 phr, at least 0.1 phr, or at least 0.5 phr. All ranges bound by any selection of the lower and upper concentrations noted above are expressly included. In some embodiments. it can be desirable for the polymer latex composition to comprise a concentration specifically in the range of about 0.1 to about 10 phr based on the weight of the polymeric component present in the polymer latex composition. In certain embodiments, the boron nitride can be present at a range of about 0.2 phr to about 9 phr, about 0.5 to about 8 phr, about 1 phr to about 7 phr, or about 2 to about 6 phr.

In some embodiments, the polymer latex composition used to prepare the articles of the present disclosure may include one or more further components in addition to the polymeric component and the boron nitride particles. The combination of the polymeric component and boron nitride with the one or more further components can be referred to as a compounded latex composition.

In one or more embodiments. one or more cure accelerators may be included polymer latex composition. Suitable cure accelerators can include. for example, one or more dithiocarbamates, and a blend of a plurality of dithiocarbamates may particularly be used. Non-limiting examples of suitable dithiocarbamates can include zinc dibutyldithiocarbamate (ZDBC), zinc diethydithiocarbamate (ZDEC). zinc dimethyldithiocarbamate (ZDMC), zinc dibenzyl dithiocarbamate (ZBED), sodium diethyl dithiocarbamate (SDEC), and sodium dibutyldithiocarbamate (SDBC). Further accelerators that may be used include those that may otherwise be known as activators or catalysts for vulcanization. For example. zinc oxide can be used. Activators or catalysts may be used in combination with other accelerators as described herein.

The polymer latex composition may include sulfur (e.g., free sulfur, such as being present in an S₈ configuration) and/or one or more sulfur donors, and such components may be recognized as curing agents for vulcanization of rubber compositions. In some instances, the sulfur donor may also be classified as or recognized in the field as being an accelerator. In some embodiments, useful sulfur donors can include one or more thiurams, such as dipentamethylenethiuram hexasulfide (DPTTH), dipentamethylenethiuram tetrasulfide (DPTT), tetramethylthiuram monosulfide (TMTM), tetramethylthiuram disulfide (TMTD), tetraethylthiuram disulfide (TETD), and tetrabenzylthiuram disulfide (TB_{z}TD). Additionally. or alternatively, other types of sulfur donors may also be utilized. For example, 4,4′-dithiodimorpholine (DTDM), thiocarbamyl sulfonamide, and N-oxydiethylene thiocarbamyl-N-oxydiethylene sulfenamide (OTOS) may be utilized in some embodiments. The use of such materials can be beneficial in that the sulfur included in the sulfur donor compounds does not contribute to potential allergies, such as can be encountered with the use of free sulfur.

A single curing accelerator or a mixture of two or more curing accelerators may be used in the polymer latex composition in a total amount based upon a composition including 100 phr of the polymeric component. For example. in some embodiments, a single curing accelerator may be used in an amount of about 0.01 to about 5.0 phr or about 0.1 to about 3.0 phr. In other embodiments, a single curing accelerator may be used in an amount of about 0.1 to about 5.0 phr, about 0.2 to about 4.5 phr, or about 0.4 to about 4.0 phr. In further embodiments, a total amount of all curing accelerators in the polymer latex composition can be about 0.2 to about 8.0 phr, about 0.4 about 5.0 phr, or about 1.0 to about 3.0 phr. In some embodiments, a sulfur donor may be considered to be a cure accelerator, and the amount of a sulfur donor may be within the above-recited ranges for single cure accelerators and/or for total cure accelerators. Alternatively, the above-discussed ranges may be applied individually to components utilized as accelerators and to components utilized as sulfur donors.

The polymer latex composition can comprise one or more additional components that can be useful. for example, to assist in resisting aging (and thus maintaining stability of the end product) and/or to provide additional, useful properties to the formed elastomeric article. As non-limiting examples. further materials that can be used are as provided below. Surfactant(s) can be used (e.g., cationic surfactants and/or anionic surfactants and/or amphoteric surfactants), and non-limiting examples include sodium lauryl sulfate, sodium polynaphthalene sulfonate, sodium polymethacrylate, and potassium laurate. Antioxidants can be used, and non-limiting examples include a butylated reaction product of p-cresol and dicylopentadiene that is available under the name Bostex™ 24 and a variety of mercapto-imidazole compounds, such as 2-mercaptobenzimidazole (MBI), 2-mercaptotoluimidazole (MTT), 2-mercapto toluimidazole (MTI), a zinc salt of 2-mercaptobenzimidazole (ZMBI), a zinc salt mercaptotoluimidazole (ZNTI), and the like. Polyphenols likewise can be utilized as antioxidants. Rheological stabilizers may be used, and non-limiting examples include a hydrophobically modified alkali swellable emulsion (“HASE”) polymer). Fillers may be used, and non-limiting examples include fumed silicas or dispersions thereof, such as available under the tradename cab-o-sperse®). Smoothing agent may be used, and these can include proteins, such as casein, and smoothing agents may likewise be referred to as stabilizers. Any of the additional components may be included in the polymer latex composition singularly, or in any combination, in an amount of about 0.01 to about 4 phr, about 0.05 to about 3.5 phr, about 0.1 to about 3.0 phr, or about 0.2 to about 2.0 phr.

In various embodiments, elastomeric articles may be prepared by conventional methods, such as dipping one or more formers into a liquid polymer composition, such as defined herein (e.g., comprising at least the polymeric component and the boron nitride particles), one or more times to form at least one layer of the polymer latex composition on the former. The boron nitride particles can be incorporated into the liquid polymer composition in a variety of manners. The boron nitride particles can be dispersed into an aqueous medium (e.g., deionized water) to form a slurry. The pre-formed slurry can be added to the liquid polymer composition during vulcanization or the pre-formed slurry can be added to the liquid polymer composition after vulcanization.

Methods of preparing a polymer latex composition and methods of preparing the elastomeric article may include a plurality of steps including mixing of polymer composition components, one or more steps wherein a former of other mold is dipped or otherwise coated with one or more coatings or layers of polymer composition to form a film of a desired thickness, and a curing step wherein the formed film is processed to be in a substantially finished form (e.g., crosslinked or otherwise solidified to form a unitary article of manufacture). Optionally, one or more drying steps may be utilized. Further, suitable processing equipment may be used as needed to provide for the necessary processing steps, including formers, dip tanks, heating equipment, fans, conveyers, and the like may be utilized.

A polymeric component may be provided as a dispersion that may be obtained from a supplier in a higher solid content than is desired for the end products. Accordingly, a method of manufacture of an elastomeric article can include diluting a polymer latex dispersion (e.g., using deionized water or the like) to the desired solid content. In some embodiments, total solids content can be in the range of about 30% to about 70%, about 34% to about 65%, about 40% to about 60%, or about 45% to about 55%. Additives. including the boron nitride particles. may be added sequentially or simultaneously to the polymer latex dispersion to form the polymer latex composition. Where surfactants and accelerators are utilized, these in particular may be added together to the polymer latex dispersion and stirred for a time to reach a substantially homogeneous dispersion of the materials. Antioxidant may specifically be added to the polymer latex composition after addition of the further components, such as within a few hours of the start of any dipping or other coating process. The polymer latex composition may be filtered prior to being transferred to a dip tank or storage tank for storage for a time suitable for de-aeration of the mixture. For example, the polymer latex composition may be filtered using a 200 μm filter (e.g., suitable to filter out particles having a size greater than 200 μm) or a differently sized filter (e.g., suitable to filter out particles having a size that is greater than 150 μm, greater than 175 μm. greater than 200 μm, or greater than 225 μm).

Methods for preparing an elastomeric article can comprise forming a film on a former or other mold using the polymer latex composition that includes the polymeric component (i.e., the polymer latex dispersion) and the boron nitride particles (and any further additives as described herein). Coating or dipping can be carried out as one or more individual coating or dipping actions to achieve the desired layer thickness of the elastomeric article. Where multiple dipping or coating steps are carried out, these may be separated by a drying period. Thus, a first coating or dipping action may be carried out to begin forming of a film, the partial film may be at least partially dried during the drying period, and a second coating or dipping action may be carried out to further form or complete forming of the film. An individual drying period may be carried out for a defined time under defined conditions. For example, a drying period may continue for a time of about 1 minute or greater, about 2 minutes or greater, or about 3 minutes or greater (such as about 1 minute to about 10 minutes or about 2 minutes to about 8 minutes). Drying conditions may be. for example, at a temperature of about 50° C. or greater, about 70° C. or greater, or about 80° C. or greater (such as about 50° C., to about 110° C., about 70° C., to about 110° C. or about 80° C., to about 110° C.). Drying may be carried out between individual coating or dipping actions and/or may be carried out after completion of all coating or dipping actions.

After all of the coating or dipping actions have been carried out, the method can further include curing the at least one layer of the polymer latex composition. In some embodiments, curing can be carried out at a temperature of about 100° C. or greater or about 110° C. or greater (such as a temperature range of about 100° C., to about 140° C. or about 110° C., to about 130° C.). The curing time can vary can be, for example, carried out for a time of about 5 minutes or greater or about 10 minutes or greater (such as about 5 minutes to about 30 minutes or about 10 minutes to about 20 minutes).

In an example embodiment, a method for preparing an elastomeric article according to the present disclosure may first comprise forming a compounded latex composition including the polymeric component and one or more further components as described herein. As noted, the boron nitride particles can be added during compounding or after compounding. As a non-limiting example, a compounded polymer latex composition can comprise the polymeric component, the boron nitride particles and optionally one or more of: a sulfur component and/or sulfur donor, at least one accelerator, one or more surfactants, one or more antioxidants, one or more fillers, and/or one or more smoothing agents. The compounded polymer latex composition may be subjected to conditions suitable for prevulcanization of the composition to a desired level of prevulcanization or crosslink density. Thereafter, a former may be dipped into the prevulcanized compounded latex composition to form at least one layer of the prevulcanized compounded latex composition thereon. In some embodiments, the former may be dipped a single time to form a single layer, or the former may be dipped twice to form two layers, or the former may be dipped three times to form three layers, or even more dipping iterations may be carried out. Where multiple dipping steps are utilized, the formed layer may be at least partially dried before carrying out the next step in the process. The layer(s) of the prevulcanized compounded latex composition may be cured to form the final elastomeric product, which them may be removed from the former using any suitable method in the field.

The elastomeric articles of the present disclosure can exhibit various properties that define the nature of the article, and such properties can arise from the intentional combination of components that achieve the desired properties. The addition of the boron nitride particles in the shape, size, and concentration ranges described herein are configured specifically to achieve the properties further defined below when the polymer latex composition is used to form an elastomeric article at a specific layer thickness.

In one or more embodiments, the characteristics defined herein may relate to an elastomeric article having an average thickness as already described above. In particular embodiments, any measured property of an elastomeric article of the present disclosure can be defined relative to the elastomeric article having a specific thickness of about 0.1 mm, such as a thickness of about 0.08 mm to about 0.12 mm. or about 0.08 mm to about 0.1 mm.

An elastomeric article according to the present disclosure in particular can exhibit a tensile strength of about 20 MPa or greater when measured in accordance with American Society for Testing and Materials (ASTM) D412. More particularly, the tensile strength can be about 22 MPa or greater. The tensile strength can be in the range of about 20 MPa to about 50 MPa, about 22 MPa to about 40 MPa ,or about 23 MPa to about 35 MPa. Likewise, an elastomeric article according to the present disclosure can exhibit a tensile modulus at 500% elongation that is less than 2.75 MPa, less than 2.25 MPa, less than 2.0 MPa, less than 1.75 MPa, or less than 1.50 MPa (such as in the range of about 0.50 to about 2.70, about 0.50 to about 2.20. about 0.75 to about 2.10, about 1.0 to about 2.0, or about 1.1 to about 1.8. Tensile strength and tensile modulus can be measured in accordance with American Society for Testing and Materials (ASTM) D412 and can be reported in relation to the dumbbell testing method and/or the ring testing method.

An elastomeric article including boron nitride particles according to the present disclosure can exhibit an increased thermal conductivity. The increase in thermal conductivity can be evaluated relative to an elastomeric article that does not include the boron nitride particles but is otherwise substantially identical in composition. The elastomeric article including the boron nitride particles can exhibit a thermal conductivity that is at least 5%, at least 10%, at least 15%, at least 20%, at least 25%, or at least 30% greater than the thermal conductivity of an elastomeric article that does not include the boron nitride particles but is otherwise substantially identical in composition. In other embodiments, the elastomeric article including the boron nitride particles can exhibit a thermal conductivity that is at least 40%, at least 50%, at least 60%, at least 75%, or at least 100% greater than the thermal conductivity of an elastomeric article that does not include the boron nitride particles but is otherwise substantially identical in composition. Although the amount of increase is not specifically limited. it is understood that the increase may have an upper limit based on the physical properties of the components used in forming the elastomeric articles. Thus. for purposes of clarity, the increase in thermal conductivity can have an upper limit of 100%, 200%, 300%, 400%, or 500%. Overall, the increase in thermal conductivity can be about 5% to about 200%, about 10% to about 150%, about 15% to about 100) %, about 20% to about 75%, or about 30% to about 50%. In other embodiments, the increase in thermal conductivity can be about 40) % to about 500) %, about 50% to about 400) %, or about 75% to about 300%. As further discussed below, in some embodiments the thermal conductivity may be as measured thru-plane or as measured in-plane. As such, any of the above ranges may be applied specifically to a thru-plane measurement or may be applied specifically to an in-plane measurement. In-plane thermal conductivity can relate to heat transfer along a surface of a tested material. and this can be tested, for example, in accordance with the method outlined in ASTM 1530. “Standard Test Method for Evaluating the Resistance to Thermal Transmission by the Guarded Heat Flow Meter Technique.” Thru-plane thermal conductivity can relate to heat transfer through the thickness of a tested material (e.g., from one surface to the opposing surface of the layer defining the elastomeric article), and this can be tested. for example, according to ISO 22007-2. “Plastics-Determination of thermal Conductivity and Thermal Diffusivity Part 2: Transient Plane Heat Source.”

In some embodiments, the present disclosure can provide a method for improving thermal conductivity of an elastomeric article (and specifically a condom). The method can comprise forming an elastomeric article from a polymer latex composition that includes boron nitride particles so as to provide the elastomeric article with at least one layer of the polymer latex composition that has the boron nitride particles distributed within. The method particularly results in an elastomeric article exhibiting a thermal conductivity that is greater than the thermal conductivity of an elastomeric article that does not include the boron nitride particles but is otherwise substantially identical in composition. The increase in thermal conductivity can be within a range that is otherwise described above. The method can specifically relate to utilization of boron nitride particles in the shapes, sizes, and concentrations otherwise described herein. In some embodiments, the method can be effective for improving thermal conductivity without reduction in tensile strength or with an increase in tensile strength. The method also can comprise compounding the polymer latex composition such that the boron nitride particles are provided as an aqueous slurry during a vulcanization step or after a vulcanization step. It is understood that vulcanization comprises crosslinking of the polymeric component with a crosslinking component (e.g., sulfur or a sulfur donor) optionally in the presence of an accelerator.

The addition of boron nitride particles in the combinations of shape, size, and concentration to a polymer latex composition as described herein can provide an elastomeric article that surprisingly exhibits improved lubricity relative to an elastomeric article that does not include the boron nitride particles but is otherwise substantially identical in composition. The lubricity of the presently disclosed elastomeric articles can be at least 5%, at least 10%, or at least 15% greater than the lubricity of an elastomeric article that does not include the boron nitride particles but is otherwise substantially identical in composition.

Lubricity can be measured on a Texture Analyzer equipped with a 40 mm small sled. The equipment contains a platform having a friction sled attached to a load cell which is constrained to slide across the platform over which a lubricant is applied. Load is provided by a 300 g weight positioned centrally over the sled. This arrangement was used to measure the coefficient of sliding friction over a fixed period of time. The Plexiglas™ sled is covered with the tested elastomeric article having a standard silicone lubricant coated thereon (e.g., 100% active silicone polymer, or dimethicone). The study generated a coefficient of friction data for an extended period of approximately fifteen (15) minutes which was translated to lubricity as in the equation provided below, where λ is the coefficient of friction. F_{f} is the force of friction. Fₙ is the normal force, and lubricity is the reciprocal of the coefficient of friction.

$$μ = {F}_{f} / {F}_{N} ; Lubricity = {μ}^{- 1}$$

Lubricity is interpreted as being the inverse of the coefficient of friction (μ). The more drag that is experienced on the surface, the lower the measured lubricity. As such, higher lubricity values are seen as evidence of improved properties according to the present disclosure.

In some embodiments, the present disclosure can provide a method for improving lubricity of an elastomeric article (and specifically a condom). The method can comprise forming an elastomeric article from a polymer latex composition that includes boron nitride particles so as to provide the elastomeric article with at least one layer of the polymer latex composition that has the boron nitride particles distributed within. The method particularly results in an elastomeric article exhibiting a lubricity that is at least 5% greater than the lubricity of an elastomeric article that does not include the boron nitride particles but is otherwise substantially identical in composition. The increase in lubricity can otherwise be within a range that is also described herein. The method can specifically relate to utilization of boron nitride particles in the shapes, sizes, and concentrations otherwise described herein. In some embodiments, the method can be effective for improving lubricity without reduction in tensile strength or with an increase in tensile strength. The method also can comprise compounding the polymer latex composition such that the boron nitride particles are provided as an aqueous slurry during a vulcanization step or after a vulcanization step. It is understood that vulcanization comprises crosslinking of the polymeric component with a crosslinking component (e.g., sulfur or a sulfur donor) optionally in the presence of an accelerator. The method also can comprise a combination of improving thermal conductivity and improving lubricity. Still further, the method can comprise a combination of improving all of thermal conductivity, lubricity, and tensile strength.

The present disclosure is more fully illustrated by the following examples. Said examples are set forth to illustrate certain embodiments of the present disclosure and are not to be construed as limiting thereof.

### Example 1—Preparation of Compounded Polymer Latex Compositions

A compounded polymer latex composition was prepared using natural rubber latex (NRL) as the polymer component. The NRL was diluted to the desired solids content (e.g., about 50% to about 55%) in deionized water. Thereafter, with mixing, stabilizer, surfactant, curing agent, catalyst, and accelerators were added. In some samples. boron nitride particles were added at this stage during compounding. The composition was allowed to sit with stirring at room temperature overnight. Antioxidant was added to the composition approximately two hours prior to preparation elastomeric articles. In some sample, the boron nitride particles were withheld during compounding and were instead added at this stage. prior to article preparation. The compounded polymer latex compositions were prepared to achieve a composition for use in preparation of condoms for mechanical testing and also for use in preparation of films for mechanical testing and lubricity testing. The formulations included 100 phr natural rubber latex. 0.01 to 0.1 phr stabilizer/smoothing agent, 0.05 to 0.2 phr surfactant. 0.5 to 3 phr curing agent. 0.1 to 0.2 catalyst, 0.01 to 0.5 phr accelerator, and 0.1 to 1 phr antioxidant. Formulations for condoms used in mechanical testing (see Examples 2-4) included boron nitride particles in amounts of 0) phr and 2.5 phr. Formulations for films used in mechanical testing (see Examples 3-4) included boron nitride particles in amounts of 0) phr, 1 phr, 2.5 phr, and 5 phr. Formulations for films used in thermal testing (see Example 5) included boron nitride particles in amounts of 0) phr and 12 phr.

### Example 2—Preparation of Condoms

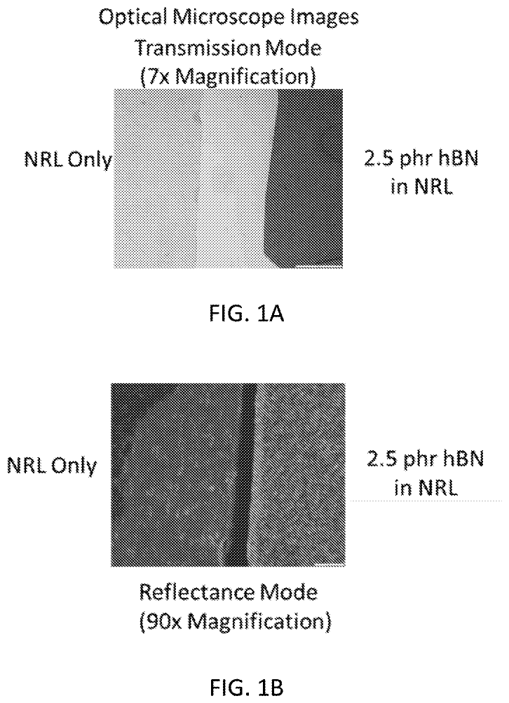

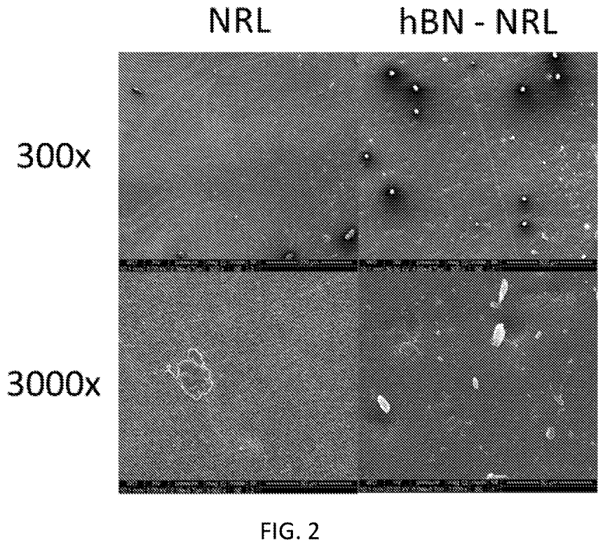

Condoms were formed by dip-molding glass formers in the compounded polymer latex compositions and heat curing the resulting film. All condoms were prepared to meet the length and film thickness requirements for a standard condom, for example about 180 mm length and about 0.08 to about 0.09 mm (80-90 microns) film thickness. Condoms were prepared by twice dipping the glass former into the polymer latex composition. The former with the two layers of the polymer latex composition was dried and then cured in an oven at standard temperatures and times (e.g., a time of about 1 minute to about 30 minutes. about 2 minutes to about 25 minutes. or about 3 minutes to about 20 minutes, and a temperature of about 100° C., to about 200° C., about 110° C., to about 180° C. or about 120° C., to about 160° C.). Following drying and curing, all condoms were submerged in a leach solution, removed from the former, washed in a corn starch slurry, and dried. Prepared condoms including the boron nitride particles were whiter in appearance than the reference condom that did not include the boron nitride particles. The differing concentration of boron nitride particles led to differences in visual appearance. Condoms appeared whiter and less transparent as boron nitride particle increased. Additionally, condoms made with boron nitride platelets appeared whiter and less transparent than condoms made with boron nitride particles in the form of agglomerates. Optical microscope evaluation showed a uniform distribution of the boron nitride particles in the elastomeric article. FIG. 1A shows a comparison in transmission mode of the rubber only article (left) with the article including boron nitride particles in a concentration of 2.5 phr (right). FIG. 1B shows a comparison in reflectance mode of the rubber only article (left) with the article including boron nitride particles in a concentration of 2.5 phr (right). There was no indication of large agglomerates in either the transmission image or the higher magnification reflectance image. In the transmission image, the light is blocked by the boron nitride particles, which causes a black/opaque appearance. The reflectance image, which was under higher magnification, showed the boron nitride particles presenting with a grainy, white appearance. which is uniformly distributed throughout the film. The uniform distribution is desirable for providing consistent heat transfer. FIG. 2 shows scanning electron microscope (SEM) images of the elastomeric article without the boron nitride particles (left) and with 2.5 phr of the boron nitride particles (right). The SEM images reveal intact boron nitride particles in the layer of the elastomeric article with a uniform distribution.

Presence of the boron nitride particles in the elastomeric article was validated using Raman spectroscopy. Born nitride has a documented spectral peak at 1366 cm-1 that corresponds to the in-plane B-N vibrational mode. This peak was observed in the raw material powder and the elastomeric article including the boron nitride particles, but not in the reference elastomeric article where no boron nitride was added. Analysis was carried out using a piece of the elastomeric article including 2.5 phr of the boron nitride particles compared to the pure boron nitride powder and compared to a piece of the reference elastomeric article with no boron nitride. The raw boron nitride powder showed a strong (approximately 30.000 a.u.) at 1366.2 cm⁻¹. The elstomeric article including 2.5 phr of the boron nitride particles also exhibited a smaller peak (approximately 4.500 a.u.) at around the same shift. No peak was observed with the reference elastomeric article with no boron nitride.

The addition of boron nitride to the polymer latex compositions was not observed to impact the viscosity of the composite emulsion. The observed average viscosity of the control polymer latex composition (no added boron nitride) was 29.1 Cp. The polymer latex composition with 2.5 phr boron nitride particles added during compounding exhibited an average viscosity of 28 Cp. The polymer latex composition with 2.5 phr boron nitride particles added after compounding exhibited an average viscosity of 23.8 Cp. The difference in viscosity observed between the test compositions was attributed to a difference in total solid percentage level. which was 1% lower (51% total solids.) for the sample where the boron nitride particles were added after compounding. Viscosity was measured using a Brookfield DVI at 30 rpm using the S61 spindle. Total solids content was measured using a Sartorius Mark 3 Moisture Analyzer. Stability of the samples was investigated by monitoring viscosity over time for 10 days for 3 levels of increasing boron nitride phr post-added to a pre-vulcanized (PV) polymer latex composition and comparing to a control sample of pre-vulcanized polymer latex composition without boron nitride. Testing showed no significant impact observed for any phr level of boron nitride in the samples as evaluated over a 10-day period.

### Example 3—Mechanical Properties of Prepared Articles

Articles prepared according to Example 2 were evaluated for various properties thereof. Testing initially was to identify any changes caused by the addition of boron nitride particles. The mechanical properties of the elastomeric articles including the boron nitride particles were not found to be negatively impacted. Surprisingly, the average tensile strength was found to be directionally higher in the elastomeric articles containing 2.5 phr boron nitride, which was added after the vulcanization process, however, there was a small loss in strain (%). Table 1 (dumbbell test method) and Table 2 (ring test method) summarize the condom mechanical testing data, which was carried out in accordance with ASTM D3492 Standard Specification for Rubber Contraceptives (Male Condoms) and used both of the dumbbell testing method (Table 1) and the ring testing method (Table 2). Tested samples were made with natural rubber latex (NRL) as control and NRL with 2.5 phr boron nitride particles (NRL +2.5 BN) as the inventive samples. The averaged results from each of the tests are provided in Table 1 and Table 2.

TABLE 1

| | Avg. | Tensile | Break | Elongation |
|---|---|---|---|---|
| Composition | Thickness (mm) | Strength (MPa) | (N) | (%) |
| NRL average | 0.0866 | 24 | 7.95 | 1905 |
| NRL + 2.5 BN | 0.08865 | 25.15 | 9.4 | 1880 |
| Average | | | | |

TABLE 2

| | Avg. | Tensile | Break | Elongation |
|---|---|---|---|---|
| Composition | Thickness (mm) | Strength (MPa) | (N) | (%) |
| NRL average | 0.0890 | 17.3 | 61.6 | 836 |
| NRL + 2.5 BN | 0.0894 | 23.4 | 83.1 | 902 |
| Average | | | | |

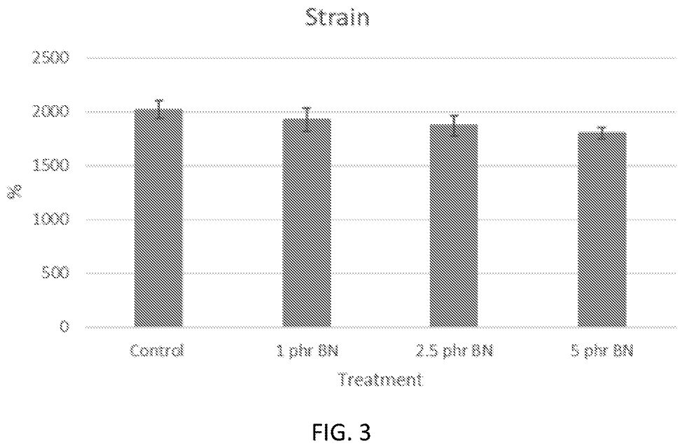

Dose response testing was carried out on the tensile and strain properties using drawn films of pre-vulcanized latex. All films were drawn using an 8 mil draw down bar on a glass plate and resulted in uniform films of about 100 microns in thickness with less than a 10% RSD in thickness. Each treatment was an average and standard deviation of 12 dumbbell latex pieces cut from 3 independent drawn films. The graph in FIG. 3 shows the decreasing trend for strain; however, at the levels tested, the strain still fell within a reasonable target (˜1800%) for typical applications of such elastomeric articles, such as condoms. The control sample was NRL without boron nitride, and the test samples were substantially identical NRL but including the indicated content of boron nitride particles.

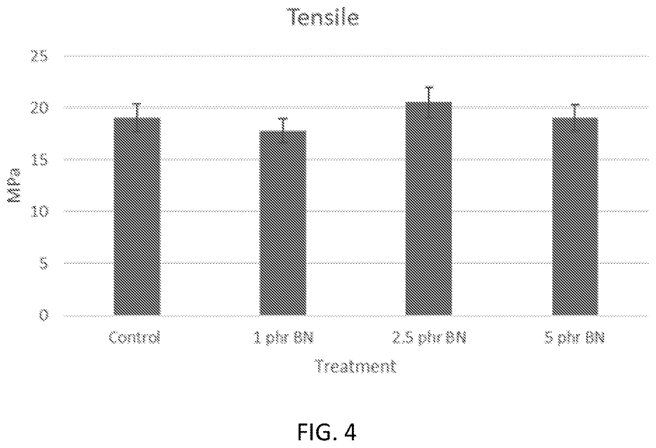

Results of the dose response testing in relation to tensile strength are shown in FIG. 4. Tensile strength was not observed to be significantly impacted with the inclusion of the boron nitride particles in the NRL. Surprisingly, the directional increase observed in the condom testing was again observed in the drawn films of equivalent thickness. This suggests that at the 2.5 phr level of inclusion, the boron nitride particles provide improvement of film tensile strength.

### Example 4—Lubricity of Prepared Articles

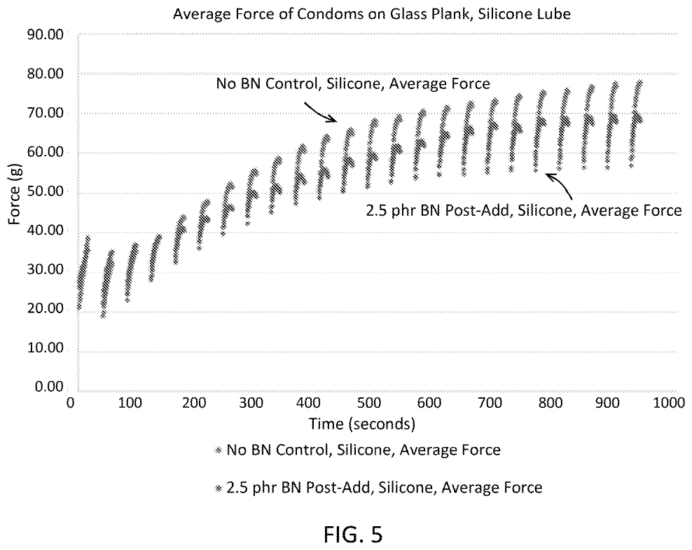

Testing indicated that the elastomeric articles including boron nitride particles exhibited improved lubricity relative to elastomeric articles that did not include boron nitride particles but were otherwise substantially identical in composition. Lubricity was measured using a method and instrument where a weighted sled was moved across a platform lubricated with a standard silicone lubricant and the amount of force required to move the sled was measured by a calibrated scale. Testing was carried out on a condom comprising a polymer latex composition that included a polymeric component (NRL) and boron nitride particles (2.5 phr added after vulcanization) and also on a condom that did not include boron nitride particles but was otherwise substantially identical in composition. Lubricity was measured on a Texture Analyzer equipped with a 40 mm small sled. The equipment contained a platform having a friction sled attached to a load cell which was constrained to slide across the platform over which a lubricant was applied. Load was provided by a 300 g weight positioned centrally over the sled. This arrangement was used to measure the coefficient of sliding friction over a fixed period of time. The Plexiglas™ sled was covered with the tested elastomeric article, and the article was coated with approximately 0.55 g of 100% active silicone polymer (dimethicone). having a viscosity of 200 centistrokes. As seen in FIG. 5, the average force required to pull the weighted sled was less for the test condom than for the control condom.

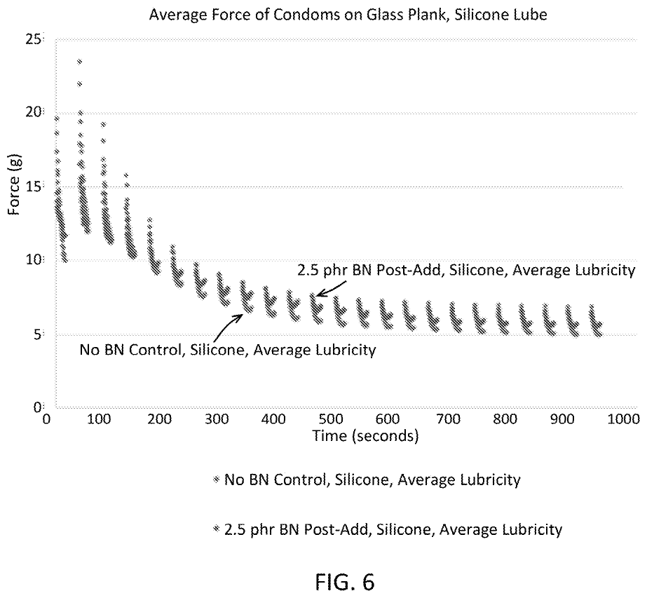

Lubricity is the measured force normalized by the total mass being pulled. FIG. 6 shows lubricity values for the date provided in FIG. 5 when force is converted to lubricity. After the transformation, it is evident that the condom comprising a polymer latex composition that included a polymeric component (NRL) and 2.5 phr boron nitride particles exhibited a higher lubricity than the control condom that did not include the boron nitride particles, as reflected by the lower required force to move the sled in the testing.

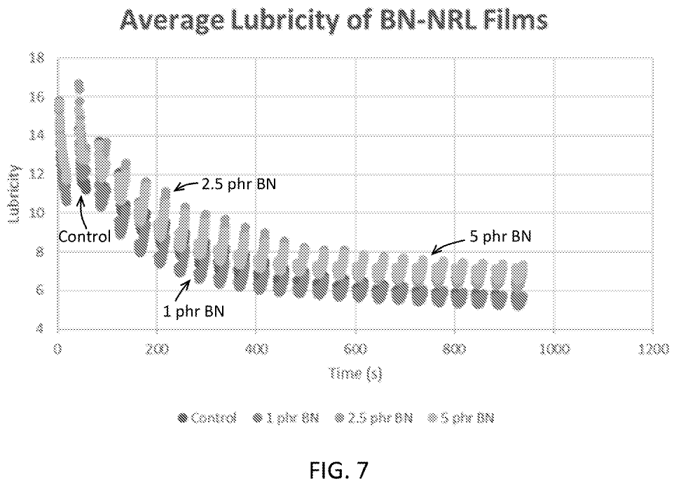

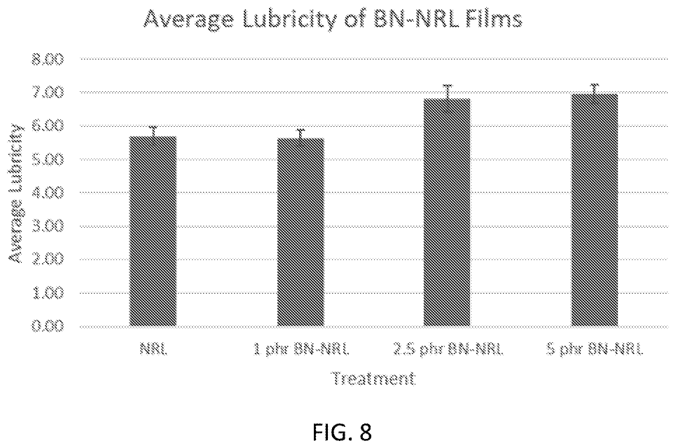

Dose response testing was also carried out in relation to lubricity, and a trend of increasing lubricity with increasing concentration of the boron nitride particles was observed using drawn thin films. Films were drawn using the same draw down bar and methods described above. The same phr levels used in the mechanical property testing were used here with levels of 1 phr, 2.5 phr, and 5 phr being compared to a control NRL film without any boron nitride particles. There was an insubstantial difference in lubricity between the control condom and the test condom with a 1 phr boron nitride additive concentration. There were significant increases in lubricity. however for the test condoms with 2.5 phr and 5 phr boron nitride additive concentrations. These results are seen in FIG. 7. The measured lubricity values exhibited achievement of a steady state toward the end of the lubricity plot as the initially deposited lubricant became spread over the sample and platform area. This steady state condition correlated to the final 300 measured values from each of the control and test condoms. Comparing these final 300 values from each treatment revealed a 20% improvement in lubricity starting at the 2.5 phr level. This improvement is statistically significant when comparing those final 300 lubricity values for the different treatments using an ANOVA at an □=0.05 (Table 4). The average lubricity values as seen in FIG. 8.

Condoms created using the polymer latex composition including boron nitride particles showed no negative interaction with lubricant, such as loss in film integrity or discoloration. This was tested using incubation of the condoms with lubricant applied for a time of four days. After the incubation period, the condoms including the boron nitride particles maintained their initial, whiter color and retained a transparency level similar NRL condom that did not include any boron nitride particles.

### Example 5—Thermal Conductivity Testing of Formed Films

Films of approximately 1 mm thick were formed in a mold using the polymer latex compositions described in Example 1. The composition was filled into the mold. heat cured, and removed from the mold. All films were prepared to meet the film thickness requirements for the thermal conductivity testing instrumentation. All films were prepared by curing the polymer latex in an oven at standard temperature and times as already noted in Example 1 and that would not damage the mold. Following drying and curing, all films were removed from the mold. washed in a corn starch slurry, and dried. Prepared films including the boron nitride particles were whiter in appearance than the reference film that did not include the boron nitride particles. Films prepared from the compositions with differing concentrations of boron nitride particles appeared whiter and less transparent as the concentration of boron nitride increased.

Elastomeric articles with the added boron nitride particles were evaluated for heat transfer properties to identify improvements in thermal conductivity. Average thermal conductivity measurements were recorded for heat transfer through the material (thru-plane) and heat transfer along a surface of the material (in-plane). Testing for thru-plane thermal conductivity was performed using a Guarded Heat Flow Meter (GFHM) method according to the method outlined in ASTM 1530. Standard Test Method for Evaluating the Resistance to Thermal Transmission by the Guarded Heat Flow Meter Technique, and in-plane thermal conductivity measurements were performed according to ISO 22007-2. Plastics—Determination of Thermal Conductivity and Thermal Diffusivity Part 2: Transient Plane Heat Source. Sample films had a thickness of approximately 1 mm were created using a mold to meet the requirements of both test methods and the measurement instrumentation.

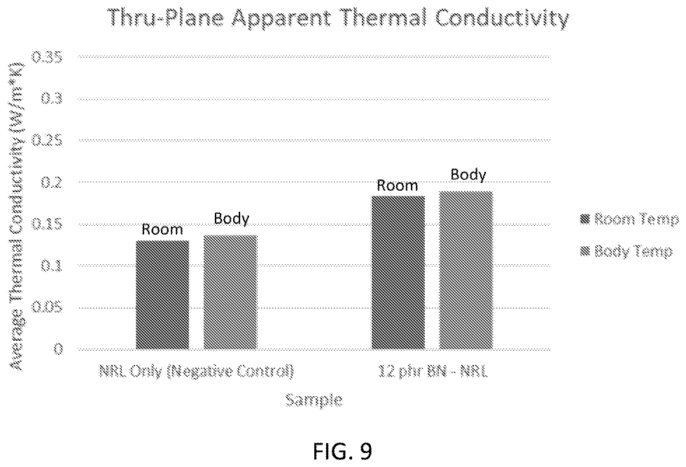

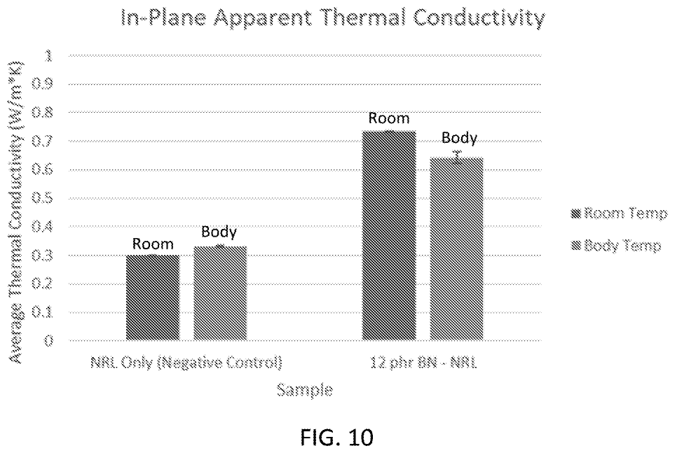

The thru-plane measurements are shown in FIG. 9 for room temperature (25° C.) and body temperature (37° C.) conditions. Heat transfer through the material was found to increase by approximately 29% at both temperature testing conditions for the samples with boron nitride particles compared to the samples without the boron nitride particles. The in-plane measurements are shown in FIG. 10 for room temperature (25° C.) and body temperature (37° C.) conditions. In-plane heat transfer was found to be at least double at both temperature testing conditions for the samples with boron nitride particles compared to the samples without the boron nitride particles. The dominance of the in-plane mode of heat transfer suggests an initially cooler feeling condom when donning. but the higher overall thru-plane thermal conductivity is evidence for a warmer condom during use.

### Example 6—Thermal Conductivity Testing of Formed Condoms

Condoms were prepared from polymer latex compositions as described in Example 1. The compositions included boron nitride in the amounts shown in Table 3.

TABLE 3

| | Comparative | Test 1 | Test 2 | Test 3 | Test 4 |
|---|---|---|---|---|---|
| Boron Nitride | (phr) | (phr) | (phr) | (phr) | (phr) |
| 1 micron platelets | 0 | 2.5 | 0 | 0 | 0 |
| 7 micron platelets | 0 | 0 | 2.5 | 5 | 0 |
| 23 micron | 0 | 0 | 0 | 0 | 2.5 |
| agglomerates | | | | | |

Film samples (four each) were cut from the prepared condoms. The thicknesses of the condom films were measured using a Mitutoyo High Accuracy Digimatic Micrometer. Each film sample was measured 4 times, totaling 16 thickness measurements per sample. Table 4 shows the average of each material.

TABLE 4

| | Avg. Thickness |
|---|---|
| Sample | (microns) |
| NRL without boron nitride (comparative) | 93.2 |
| NRL + 2.5 phr of 1 micron boron nitride platelets | 92.5 |
| NRL + 2.5 phr of 7 micron boron nitride platelets | 92.1 |
| NRL + 2.5 phr of 23 micron boron nitride agglomerates | 137.3 |
| NRL + 5 phr of 7 micron boron nitride platelets | 123.5 |

Before testing, materials were conditioned in an oven at 37° C. for 1 hour. All samples were tested in an oven at this temperature, using a 13 mm Flex TPS (Transient Plane Source) sensor and a C-Therm Trident controller (i.e., a modular system using different sensor configurations to accommodate a wide range of sample types). When the controller is operated with a TPS sensor, it is an integrated solution for thermal conductivity and thermal diffusivity measurements and operates in compliance with ISO 22007. The testing standard required an initial zero-thickness measurement only using the 304L stainless steel pucks. After a 5-minute delay, one layer of the thin film was placed on either side of the TPS sensor and sandwiched between the stainless steel pucks. The thermal resistance measurement was taken at 0.1 W of power over 4 seconds. A ΔT measurement was plotted against the thickness of the film. This process was repeated until all layers had been tested. By plotting ΔT vs thickness of film, linear regression analysis was completed, and thermal conductivity (k) was reported. Measurements are shown in Table 5 below. As seen therein, all samples including the boron nitride particles showed an improvement over the comparative. Use of boron nitride particles in platelet provided improved results versus agglomerates, and improvement also increased with increasing particle size and particle concentration.

TABLE 5

| | Avg. Thermal | Improvement |
|---|---|---|
| | Conductivity | Relative |
| Sample | (W/mK) | Comparative |
| NRL without boron nitride (comparative) | 0.148 | NA |
| NRL + 2.5 phr of 1 micron boron nitride | 0.165 | 11% |
| platelets | | |
| NRL + 2.5 phr of 7 micron boron nitride | 0.167 | 13% |
| platelets | | |
| NRL + 2.5 phr of 23 micron boron nitride | 0.154 | 4% |
| agglomerates | | |
| NRL + 5 phr of 7 micron boron nitride | 0.168 | 14% |
| platelets | | |

### Example 7—Thermal Conductivity Testing of Comparative and Inventive Condoms

The thermal conductivity of boron nitride (BN) enriched condoms was measured and compared to several commercially available condoms. Testing was carried out using the C-Therm Trident Controller. Three replicates of each condom type, unlubricated, were analyzed for thermal conductivity. Condom A was a natural rubber latex (NRL) condom sold under the brand Durex Air and having an approximate average thickness of 40.71 microns. Condom B was a polyisoprene (PI—non-latex) condom sold under the brand Durex RealFeel™ and having an approximate average thickness of 70.97 microns. Condom C was a polyisoprene (PI—non-latex) condom sold under the brand Lifestyles Skyn®) and having an approximate average thickness of 58.77 microns. Condom D was a NRL condom sold under the brand Trojan™ ENZ™ and having an approximate average thickness of 58.66 microns. Condom E was an inventive condom formed from NRL, including the BN particles, and having an approximate average thickness of 55.82 microns.

The C-Therm Trident is a modular system that uses different sensor configurations to accommodate a wide range of sample types. When operated with a Transient Plane Source (TPS) sensor, it is an integrated solution for thermal conductivity and thermal diffusivity measurements and operates in compliance with ISO 22007. The following testing parameters were used by to take the measurements.
Test Method: TPS Thin Film

Accessory: (TPS) Testing ClampSensor Size: 13 mm
Temperature Condition: 37° C.
Atmospheric Condition: Ambient
Number of Layers: 2

Each test condom was cut into 12 film samples. The thicknesses of the films were measured using a Mitutoyo High Accuracy Digimatic Micrometer, and the thickness of 5 of the 12 film samples for each condom were tested to obtain an effective thickness measurement for each condom. Table 6 shows the average thickness of each individual sample in a set and an overall average for the condom type. Before testing, materials were conditioned in an oven to 37° C. for one hour. All samples were tested in a Binder laboratory oven at the same temperature, 37° C., using a 13 mm Flex TPS sensor and a C-Therm Trident controller.

TABLE 6

| | | | | | | | | | Condom | |
|---|---|---|---|---|---|---|---|---|---|---|
| | Condom | | Condom | | Condom | | Condom | | E | |
| | A | | B | | C | | D | | (NRL + 5 | |
| | (NRL) | | (PI) | | (PI) | | (NRL) | | phr BN) | |
| Sample 1 | 43.54 | μm | 70.22 | μm | 62.84 | μm | 62.50 | μm | 54.64 | μm |
| Sample 2 | 37.74 | μm | 74.42 | μm | 55.66 | μm | 55.66 | μm | 54.32 | μm |
| Sample 3 | 40.86 | μm | 68.26 | μm | 57.82 | μm | 57.82 | μm | 58.50 | μm |
| Average | 40.71 | μm | 70.97 | μm | 58.77 | μm | 58.66 | μm | 55.82 | μm |
| Thickness | | | | | | | | | | |
| Standard | 2.90 | μm | 3.15 | μm | 3.68 | μm | 3.50 | μm | 2.33 | μm |
| Deviation | | | | | | | | | | |

The ISO testing standard requires an initial zero-thickness measurement only using 304L stainless steel pucks. For this, the sensor was sandwiched between the stainless-steel pucks and the sample assembly was left to condition in this configuration for a period of 3 minutes. The thermal resistance measurement was taken at 0.1 W of power over 4 seconds and a ΔT (temperature difference) measurement was plotted against the thickness of the film. Next, one layer of the thin film was placed on either side of the TPS sensor and sandwiched between the stainless-steel pucks, and the sample assembly was also left to condition before taking the thermal resistance measurement at the same 0.1 W of power over 4 seconds. Again, the ΔT measurement was plotted against the thickness of the film. This process was repeated with an additional layer to meet the testing standard requirements. After plotting ΔT vs the thickness of film a linear regression analysis was completed as described in ISO 22007-2, and thermal conductivity (k) was reported. All samples were processed in the same manner.

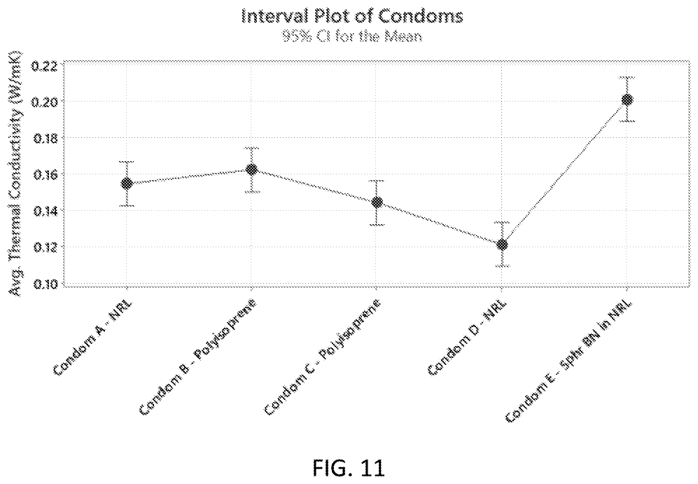

Thermal conductivity measurements are shown in Table 7, and it can be observed that adding the BN to the NRL resulted in superior heat transfer when compared to the other condoms. On average, the 5 phr BN-NRL condom (Condom E) was observed to increase thermal conductivity between 23% and 65% depending on material properties (such as, type and quality) and any processing impacts (for example, removal of residual compounding ingredients). Surprisingly, at this level the thermal conductivity of Condom E was greater than Condom A, which was more than 20% thinner. This improvement in thermal conductivity due to BN enrichment is also shown in FIG. 11.

TABLE 7

| | | | | | Condom E |
|---|---|---|---|---|---|
| | Condom A | Condom B | Condom C | Condom D | (NRL + 5 |
| | (NRL) | (PI) | (PI) | (NRL) | phr BN) |
| Sample 1 | 0.167 W/mK | 0.156 W/mK | 0.146 W/mK | 0.124 W/mK | 0.199 W/mK |
| Sample 2 | 0.144 W/mK | 0.174 W/mK | 0.136 W/mK | 0.118 W/mK | 0.190 W/mK |
| Sample 3 | 0.152 W/mK | 0.156 W/mK | 0.150 W/mK | 0.121 W/mK | 0.213 W/mK |
| Average | 0.154 W/mK | 0.162 W/mK | 0.144 W/mK | 0.121 W/mK | 0.201 W/mK |
| Conductivity | | | | | |
| Standard | 0.012 W/mK | 0.010 W/mK | 0.007 W/mK | 0.003 W/mK | 0.012 W/mK |
| Deviation | | | | | |

The data was analyzed using a One-Way Analysis of Variance (ANOVA) to determine statistical significance of the observed differences in thermal conductivity, followed by the Fisher LSD Method for a pairwise comparison, both at 95% level of confidence. All statistical analysis was performed using MiniTab software. Because the calculated p-value (p=0) is below the level of significance (a=0.05), it can be inferred that the differences in thermal conductivity are statistically significant.

The results of the pairwise analysis of the means comparing the 5 condom samples are shown in Table 8. Condom E, the 5 phr BN-NRL condom, has the highest mean thermal conductivity value and is considered significantly different from the rest of the samples tested.

TABLE 8

| | | Mean | |
|---|---|---|---|
| Sample | N | Conductivity | Grouping |

| | | | | | | |
|---|---|---|---|---|---|---|
| Condom E | 3 | 0.20067 | A | | | |
| Condom B | 3 | 0.16200 | | B | | |
| Condom A | 3 | 0.15433 | | B | C | |
| Condom C | 3 | 0.14400 | | | C | |
| Condom D | 3 | 0.12100 | | | | D |

In Table 8, means that do not share a letter are considered to be significantly different from other groups. The foregoing testing thus illustrated that, by including BN particles in an elastomeric article, such as a natural rubber latex condom, heat transfer can be significantly improved to enable a more natural sexual experience than condoms already commercially available, and this effect brings partners closer together by improving the sensation of being able to feel their partners body warmth.

The terms “about” or “substantially” as used herein can indicate that certain recited values or conditions are intended to be read as encompassing the expressly recited value or condition and also values that are relatively close thereto or conditions that are recognized as being relatively close thereto. For example, unless otherwise indicated herein, a value of “about” a certain number or “substantially” a certain value can indicate the specific number or value as well as numbers or values that vary therefrom (+or −) by 2% or less. Similarly, unless otherwise indicated herein, a condition that substantially exists can indicate the condition is met exactly as described or claimed or is within typical manufacturing tolerances or would appear to meet the required condition upon casual observation even if not perfectly meeting the required condition. In some embodiments, the values or conditions may be defined as being express and, as such, the term “about” or “substantially” (and thus the noted variances) may be excluded from the express value.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. An elastomeric article comprising at least one layer of a polymer latex composition that includes a polymeric component and boron nitride particles, wherein the elastomeric article at a thickness of about 0.1 mm or less exhibits a tensile strength of about 20 MPa or greater when measured in accordance with ASTM D412 and exhibits a thermal conductivity that is at least 5% greater than the thermal conductivity of an elastomeric article that does not include the boron nitride particles but is otherwise substantially identical in composition.

2. The elastomeric article of claim 1, wherein the elastomeric article is a condom.

3. The elastomeric article of claim 1, wherein the polymeric component comprises natural rubber.

4. The elastomeric article of claim 1, wherein the polymeric component comprises a synthetic polymer.

5. The elastomeric article of claim 4, wherein the polymeric component comprises a polyisoprene or polystyrene-polyisoprene-polystyrene (SIS) copolymer.

6. The elastomeric article of claim 1, wherein the boron nitride particles are configured as platelets.

7. The elastomeric article of claim 6, wherein the boron nitride particles have a length and a width that are each in the range of about 1 micron to about 25 microns.

8. The elastomeric article of claim 1, wherein the boron nitride particles are configured as spheroids.

9. The elastomeric article of claim 8, wherein the boron nitride particles have an average size of about 1 micron to about 25 microns.

10. The elastomeric article of claim 1, wherein the polymer latex composition comprises about 0.1 to about 20 parts per hundred (phr) of the boron nitride particles based on the weight of the polymeric component.

11. The elastomeric article of claim 1, wherein the boron nitride particles are substantially uniformly distributed in the at least one layer of the polymer latex composition.

12. The elastomeric article of claim 1, wherein the tensile strength of the elastomeric article is greater than a tensile strength of an elastomeric article that does not include the boron nitride particles but is otherwise substantially identical in composition.

13. The elastomeric article of claim 1, wherein the elastomeric article exhibits a lubricity that is greater than the lubricity of an elastomeric article that does not include the boron nitride particles but is otherwise substantially identical in composition.

14. The elastomeric article of claim 1, wherein the thermal conductivity is a thru-plane thermal conductivity measured according to ASTM 1530.

15. The elastomeric article of claim 1, wherein the thermal conductivity is an in-plane thermal conductivity measured according to ISO 22007-2.

16. The elastomeric article of claim 1, wherein the polymer latex composition is compounded with one or both of a crosslinking agent and a cure accelerator.

17. The elastomeric article of claim 1, wherein the polymer latex composition further comprises one or more of a surfactant, an antioxidant, a viscosity modifier, a filler, and a smoothing agent.

18. A method for improving thermal conductivity of a condom, the method comprising forming the condom from a polymer latex composition that includes boron nitride particles so as to provide the condom with at least one layer of the polymer latex composition that has the boron nitride particles distributed within, the condom exhibiting a thermal conductivity that is at least 5% greater than the thermal conductivity of a condom that does not include the boron nitride particles but is otherwise substantially identical in composition.

19. The method of claim 18, wherein the boron nitride particles are included in the polymer latex composition in a substantially platelet-shaped form.

20. The method of claim 18, wherein the boron nitride particles are included in the polymer latex composition in a substantially spheroid shape.
